(19) United States
(12) Patent Application Publication
(10) Pub. No.: US 2017/0204443 A1
(43) Pub. Date: Jul. 20, 2017

(54) BIOTECHNOLOGICAL PRODUCTION OF LNT, LNNT AND THE FUCOSYLATED DERIVATIVES THEREOF

(71) Applicant: BASF SE, Ludwigshafen (DE)

(72) Inventors: Florian BAUMGÄRTNER, Stuttgart (DE); Georg A. SPRENGER, Backnang (DE); Christoph ALBERMANN, Stuttgart (DE)

(21) Appl. No.: 15/324,309

(22) PCT Filed: Apr. 10, 2015
(86) PCT No.: PCT/EP2015/057805

§ 371 Date: Jan. 6, 2017

(30) Foreign Application Priority Data
Jul. 14, 2014 (EP) 14176958.8
Dec. 18, 2014 (EP) 14198960.8

(51) Int. Cl. C12P 19/26; C12N 9/90; C12N 9/12; C12N 9/10; C12P 19/18; C12N 15/52

(52) U.S. Cl. CPC C12P 19/26; C12P 19/18; C12Y 204/01146; C12Y 204/01062; C12Y 204/01022; C12Y 207/07064; C12Y 501/03002; C12Y 207/0703; C12Y 207/01052; C12N 15/52; C12N 9/1241; C12N 9/1051; C12N 9/90

(57) ABSTRACT

The present invention relates to primarily genetically modified microorganisms for in vivo synthesis of lacto-N-tetrose (LNT) and lacto-N-neotetrose (LNnT), and their fucosylated derivatives, and to uses of such microorganisms in methods of producing lacto-N-tetrose and lacto-N-neotetrose, and their fucosylated derivatives.

The present invention relates to genetically modified microorganisms for in vivo synthesis of lacto-N-tetrose (LNT) and lacto-N-neotetrose (LNnT), and their fucosylated derivatives, and to uses of such microorganisms in methods of producing lacto-N-tetrose and lacto-N-neotetrose, and their fucosylated derivatives.

Human breast milk is considered to have an important role in healthy infant development. The oligosaccharides present therein (human milk oligosaccharides (HMO)) are one of the major constituent components of breast milk, and their core structure has a lactose unit at the reducing end and is continued with N-acetyllactosamine units in a branched or chain-like manner. Structural variability is additionally expanded by fucosyl or sialyl modifications at the terminal positions.

Lacto-N-tetrose (LNT) is a tetrasaccharide of the chemical formula N-[(2S,3R,4R,5S,6R)-2-{[(2R,3S,4S,5R,6S)-3,5-dihydroxy-2-(hydroxymethyl)-6-{[(2R,3S,4R,5R)-4,5,6-trihydroxy-2-(hydroxymethyl)oxan-3-yl]oxy}oxan-4-yl]oxy}-5-hydroxy-6-(hydroxymethyl)-4-{[(2R,3R,4S,5R,6R)-3,4,5-trihydroxy-6-(hydroxymethyhoxan-2-yl]oxy}oxan-3-yl]acetamide having the following structure:

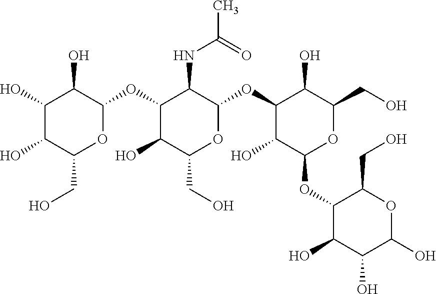 [SMILES: CC(=O)N[C@H]1[C@H](O[C@H]2[C@@H](O)[C@@H](CO)O[C@@H](O[C@H]3[C@H](O)[C@@H](O)C(O)O[C@@H]3CO)[C@@H]2O)O[C@H](CO)[C@@H](O)[C@@H]1O[C@@H]1O[C@H](CO)[C@H](O)[C@H](O)[C@H]1O]

Lacto-N-neotetraose (LNnT) has the chemical formula N-[(2S,3R,4R,5S,6R)-2-{[(2R,3S,4S,5R,6S)-3,5-dihydroxy-2-(hydroxymethyl)-6-{[(2R,3R,4R,5R)-1,2,4,5-tetrahydroxy-6-oxonexan-3-yl]oxy}oxan-4-yl]oxy}-4-hydroxy-6-(hydroxymethyl)-5-{[(2S,3R,4S,5R,6R)-3,4,5-trihydroxy-6-(hydroxymethyl)oxan-2-yl]oxy}oxan-3-yl]acetamide and the following structure:

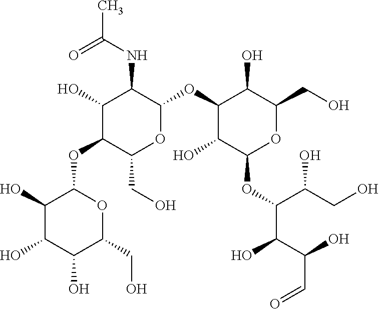 [SMILES: CC(=O)N[C@H]1[C@H](O[C@H]2[C@@H](O)[C@@H](CO)O[C@@H](O[C@@H]([C@H](O)[C@@H](O)C=O)[C@H](O)CO)[C@@H]2O)O[C@H](CO)[C@@H](O[C@@H]2O[C@H](CO)[C@H](O)[C@H](O)[C@H]2O)[C@@H]1O]

With regard to health and development promoting action, the biological function of HMOs has been the subject of numerous studies, although this requires the recovery of pure compounds in sufficient quantities. Currently, the most commonly used method is the rather complicated extraction from breast milk. Biotechnological methods of producing HMOs have been described (see Han et al., Biotechnol. Adv. 2012, 30, 1268-1278), but lacto-N-tetrose, for example, as one of the most common HMOs, is currently not available for research at a reasonable price. Both chemical and enzymatic syntheses for LNT are known from the literature (see Aly et al., Carbohydr. Res. 1999, 316, 121-132; Murata et al., Glycoconj. J. 1999, 16, 189-195). However, since chemical synthesis requires multiple steps of protection and deprotection of reactive groups and enzymatic synthesis suffers from unfavorable equilibrium product distributions and regioselectivities, these methods do not produce any satisfactory results.

It was therefore a primary object of the present invention to specify a system, preferably microorganisms, which is/are capable of producing high yields of LNT and LNnT and of their fucosylated derivatives.

Another object of the present invention was to provide a corresponding method enabling LNT and LNnT and their fucosylated derivatives to be biotechnologically produced in an efficient and inexpensive manner.

The primary object is achieved according to the invention by a genetically modified microorganism for in vivo synthesis of lacto-N-tetrose, said microorganism comprising

(i) a first transgene coding for β1,3-N-acetylglucosaminyltransferase, and

(ii) a second transgene coding for β1,3-galactosyltransferase.

According to a further embodiment, the present invention relates to a genetically modified microorganism for in vivo synthesis of lacto-N-neotetrose, said microorganism comprising

(i) a first transgene coding for β1,3-N-acetylglucosaminyltransferase, and

(ii) a second transgene coding for β1,4-galactosyltransferase.

A genetically modified microorganism in the present context means a microorganism in which individual genes have been switched off and/or endogenous or exogenous genes have been incorporated (transgenes) in a specific manner using biotechnological methods. A transgene in accordance with the present invention may be a gene imported from a different organism or else a gene which is naturally present in the microorganism concerned that has been integrated by genetic engineering at a different site in the genome and as a result is expressed, for example, under a promoter different from the natural promoter.

Surprisingly, it was found in the course of the present invention that micro-organisms routinely employed in genetic engineering, which transgenically express β1,3-N-acetylglucosaminyltransferase and a β1,3-galactosyltransferase or β1,4-galactosyltransferase, can successfully be employed in the synthesis of LNT and LNnT, respectively. It is possible to use here, for example, Leloir glycosyltransferases (LgtA or LgtB) which firstly react lactose as substrate for glycosylation to give lacto-N-triose II (LNT II) as intermediate and then, in a step dependent on nucleotide-activated sugars, elongate it to give LNT (see FIG. 1 and Frey et al. FASEB J. 1996, 10, 461-70). Examples of transgenes with proven suitability within the scope of the invention are the Neisseria meningitides IgtA gene coding for β1,3-N-acetylglucosaminyltransferase and the E. coli wbgO gene coding for β1,3-galactosyltransferase. The donor substrates of the recombinant glycosyltransferases LgtA (UDP-N-acetylglucosamine) and WbgO (UDP-galactose) are intermediates of the E. coli K12 metabolism and are continuously synthesized during growth (see Raetz et al., Annu. Rev. Biochem., 2002, 71, 635-700). UDP-N-Acetylglucosamine is a precursor of the peptidoglycan, lipopolysaccharide and enterobacterial common antigen biosyntheses (see Neidhardt et al., Cellular and Molecular Biology, second edition 1996). It is produced from fructose 6-phosphate by the GlmS, GlmM, and GlmU biosynthesis enzymes (see Barreteau et al., FEMS Microbiol. Rev., 2008, 32, 168-207). UDP-Galactose is a precursor substrate of lipopolysaccharide biosynthesis and colanic acid biosynthesis in E. coli and is formed from glucose 6-phosphate in three enzymatic steps catalyzed by Pgm, Galli, and GalE (see Frey, FASEB J., 1996, 10, 461-70). For cell growth and intracellular provision of said nucleotide-activated sugars, inexpensive substrates such as glycerol or glucose may advantageously be employed.

According to a preferred embodiment of the present invention, the microorganism is in addition genetically modified so as to suppress expression of LacZ and LacA. In a particularly preferred embodiment, the (i) first transgene here has been integrated into the LacZYA locus and the microorganism comprises a further transgene coding for LacY.

In order to prevent metabolism and possible acetylation of lactose by LacZ and LacA, expression of these genes may be suppressed in a microorganism of the invention. This is preferably performed by integrating the first transgene (i) into the LacZYA locus. However, in order to ensure that the microorganism will still take up lactose, LacY, in a preferred embodiment, is expressed transgenically at a different site in the genome, for example under a different promoter, preferably a P_{tac} promoter. Particularly preferably, lacY is integrated into the fucIK locus coding for fucose metabolism genes.

To ensure LNT and LNnT yields are as high as possible, it is advantageous to provide plenty of nucleotide-activated sugars, in particular UDP-galactose, intracellularly for conversion of LNT II to LNT to be able to proceed efficiently.

Accordingly, in a further preferred embodiment of the present invention, the microorganism comprises a further transgene coding for a UDP-sugar pyrophosphorylase (USP).

is Such a USP is encoded, for example, by the LmjF17.1160 open reading frame in Leishmania major (see Damerow et al., J. Biol. Chem. 2010, 285, 878-887). Said USP catalyzes generation of UDP-galactose utilizing galactose 1-phosphate. Advantageously, this reaction may also prevent a possibly cytotoxic accumulation of galactose 1-phosphate.

In a further preferred embodiment of the present invention, the microorganism is in addition genetically modified so as to suppress expression of UDP-glucose 4-epimerase.

This kind of suppression may be achieved, for example, by deleting the galE-gene which preferably is replaced with a T5 promoter for continued expression of the downstream genes of the operon. Advantageously, the intracellular UDP-galactose concentration is likewise increased as a result. Particular preference is given to a combination of this embodiment with the microorganism comprising a transgene coding for a UDP-sugar pyrophosphorylase (USP) (as described above).

Particular preference according to the invention is furthermore given to a microorganism of any of the embodiments described above, in which one or both, or one, multiple or all, transgenes is/are chromosomally integrated.

Using a plasmid-free strain is particularly advantageous, since maintaining productivity does not require any selection pressure (antibiotic resistances). Moreover, the use of antibiotics in food-related or pharmaceutically applicable production processes is not desirable.

According to another preferred embodiment of the microorganism of the invention, said microorganism comprises a further transgene coding for a bifunctional enzyme having L-fucokinase activity and L-fucose-1-phosphate guanylyltransferase activity, and at least one transgene coding for an enzyme capable of α(alpha)1,2-fucosylation, a(alpha)1,3-fucosylation or α(alpha)1,4-fucosylation.

Such a microorganism is capable of producing, by way of a reaction following synthesis of LNT or LNnT, the fucosylated derivatives of these two compounds, thus expanding the possible applications of the microorganism of the invention with regard to structural variability of the naturally occurring HMOs (see FIG. 2). For example, FKP may be employed as a bifunctional enzyme having L-fucokinase activity and L-fucose-1-phosphate guanylyltransferase activity. Suitable for fucosylation, for example, is expression of the enzymes encoded by the futC (α1,2-fucosylation), fucT14 (α1,4-fucosylation) or futA (α1,3-fucosylation) genes.

In a preferred embodiment of the microorganism of the invention (as described above), the transgene coding for said bifunctional enzyme having L-fucokinase activity and L-fucose-1-phosphate guanylyltransferase activity is chromosomally integrated, and the at least one transgene coding for an enzyme capable of α1,2-fucosylation, α1,3-fucosylation or α1,4-fucosylation is expressed on a plasmid vector.

In a particularly preferred embodiment of the microorganism of the invention (as described above), both the transgene coding for said bifunctional enzyme having L-fucokinase activity and L-fucose-1-phosphate guanylyltransferase activity and the at least one transgene coding for an enzyme capable of α1,2-fucosylation, α1,3-fucosylation or α1,4-fucosylation are chromosomally integrated.

A further aspect of the present invention relates to the use of a genetically modified microorganism as described herein, preferably as described as preferred herein according to any of the embodiments described above, for in vivo synthesis of lacto-N-tetrose or lacto-N-neotetrose or a fucosylated derivative of lacto-N-tetrose or lacto-N-neotetrose.

The use of such a genetically modified microorganism enables lacto-N-tetrose or lacto-N-neotetrose or a fucosylated derivative of lacto-N-tetrose or lacto-N-neotetrose to be produced efficiently and inexpensively on a scale that can be adapted to the intended use.

According to a further aspect, the present invention relates to a method of preparing lacto-N-tetrose or lacto-N-neotetrose or a fucosylated derivative of lacto-N-tetrose or lacto-N-neotetrose, comprising the following steps:
(a) providing a genetically modified microorganism as described above, preferably as described as preferred above,
(b) culturing said genetically modified microorganism under conditions that permit synthesis of lacto-N-tetrose and lacto-N-neotetrose,
(c) optionally adding fucose,
(d) optionally isolating the synthesized lacto-N-tetrose or lacto-N-neotetrose or fucosylated derivative of lacto-N-tetrose or lacto-N-neotetrose.

The method of the invention comprises firstly providing a genetically modified microorganism as described above and culturing thereof for example in a shaker flask under conditions that permit synthesis of lacto-N-tetrose and lacto-N-neotetrose. Cell growth here depends primarily on the microorganism employed. Preferably, the microorganism employed is a microorganism routinely used for biotechnological applications which has been optimized for maximum productivity. Aside from the essential lactose, inexpensive (further) carbon sources may advantageously be used, for example selected from the group consisting of glucose, glycerol, galactose, and any mixtures thereof. To allow synthesis of LNT or LNnT, lactose must be present as substrate, and expression of transgenes (i) and (ii) (as described above) must be induced, optionally as a function of the promoter under which they are expressed. To ensure fucosylation of the products, fucose must also be added. Fucose is added preferably only after induction of the genes for LNT or LNnT synthesis, ideally in such a way that sufficient quantities of appropriate substrate are present and not that only lactose is fucosylated. Alternatively, fucose may already be present at the start of step (b), and expression may be put under a promoter different from the one regulating expression of the genes for LNT or LNnT synthesis. The fucosyltransferase genes are then induced by adding the appropriate inducer at the desired time. In a preferred development, the genes for LNT or LNnT synthesis are expressed under an IPTG-inducible promoter and the fucosyltransferase genes are expressed under a rhamnose-inducible promoter in this case.

Optionally, the products produced are then isolated. For this, the cells are collected by means of centrifugation, for example, resuspended in water and lysed. The produced sugars may then be purified from the supernatant using standard methods.

In a preferred embodiment of the method of the invention, step (b) comprises
using galactose as carbon source for said microorganism, or
using glycerol and galactose as carbon source for said microorganism.

In the course of the present invention, it has been found that the yield of LNT in relation to the LNT II intermediate can be controlled via the carbon sources provided (see FIG. 3). Accordingly, particularly high yields arise, for example, when galactose is the primary carbon source present. Preferably, the (weight) proportion of galactose, in relation to the total weight of the lactose required for synthesis and possibly other carbon sources present, such as glycerol or glucose for example, is at least 50%, preferably 70%, particularly preferably at least 90%. Likewise, particularly high yields are achieved, when the primary carbon source used is glycerol and galactose is added at the start of induction of the genes for LNT or LNnT synthesis. Again the (weight) proportion of glycerol, in relation to the total weight of the lactose required for synthesis and possibly other carbon sources present, such as glucose for example, is at least 50%, preferably at least 70%, particularly preferably at least 90%.

Preference is furthermore given to a method of the invention (as described above) in which step (b) comprises adding one or more carbon source(s), preferably selected from the group consisting of lactose, glucose, glycerol, galactose, and any mixtures thereof, preferably at least lactose, particularly preferably lactose and galactose or lactose, galactose and glycerol, continuously or in batches.

Advantageously, possibly cytotoxic accumulations or unwanted inhibitions may be avoided by adding the particular carbon source(s) continuously or in batches, when they have been used up either completely or to a certain degree.

Preferably, the genetically modified microorganism (as described above) or the microorganism to be employed according to any use described herein or in any method described herein according to the invention is selected from the group consisting of bacteria, fungi, and plants, preferably microorganisms of the genera Corynebacterium, in particular Corynebacterium glutamicum, Bevibacterium, in particular Bevibacterium flavum, Bacillus, Saccharomyces, and Escherichia, in particular E. coli.

The use of microorganisms routinely employed in genetic engineering is particularly advantageous for conducting the present invention, because they have been optimized for high productivity and genetic engineering methods for introducing transgenes and induction of the latter are known.

In the course of the present invention, it has been demonstrated that it is possible to efficiently carry out the method of the invention in the form of a fed batch process also on a liter scale (see example 3). Preference is therefore given to a method as described above, with said method being carried out by way of a fed batch process with a batch volume in the range from 2 to 30 L, preferably from 3 to 20 L, particularly preferably from 5 to 15 L.

The invention will be explained in more detail by way of example below on the basis of figures and examples.

### BRIEF DESCRIPTION OF THE FIGURES

FIG. 1: Diagram of the intracellular reaction of lactose to give lacto-N-tetrose.

FIG. 2. Diagram of the intracellular synthesis of fucosylated HMOs with LNT as core structure. FucT is a fucosyltransferase, and LNFX are the products resulting therefrom.

FIG. 3: Proportion of the particular oligosaccharide in shaker flask cultures in the culture supernatant of the total amount of said oligosaccharide 24 hours after induction as a function of the carbon sources. Induction with 0.5 mM IPTG and addition of 2 g L⁻¹ lactose and 2 g L⁻¹ of the carbon source listed second in each case, and incubation at 30° C. and 90 rpm.

FIG. 4: LNT concentrations in shaker flask cultures 24 hours after induction as a function of the carbon sources. Induction with 0.5 mM IPTG and addition of 2 g L⁻¹ lactose and 2 g L⁻¹ of the carbon source listed second in each case, and incubation at 30° C. and 90 rpm.

FIG. 5: LNT II concentrations in shaker flask cultures 24 hours after induction as a function of the carbon sources. Induction with 0.5 mM IPTG and addition of 2 g L⁻¹ lactose and 2 g L⁻¹ of the carbon source listed second in each case, and incubation at 30° C. and 90 rpm.

FIG. 6: Structure of LNF I (LNT with an α1,2-linked fucosyl residue on the galactosyl residue at the non-reducing end).

FIG. 7: Structure of LND II (LNT with an α1,4-linked fucosyl residue on the N-acetylglucosaminyl residue and an α1,3-linked fucosyl residue on the glycosyl residue at the reducing end).

FIG. 8: Comparison of lactose consumption and product formation in the shaker bottle experiments on various carbon sources 24 hours after induction. a) Concentrations of lactose (white), LNT II (gray) and LNT (black). b) Product yields per biomass. c) Proportion of products in the culture supernatant in %.

FIG. 9: Intracellular concentration of UDP sugars during exponential growth on various carbon sources: UDP-glucose (gray), UDP-galactose (black), UPD-acetylglucosamine (white). Values are given as means and SE for ≧2 independent experiments.

FIG. 10: LNT fed batch production. Vertical, dashed lines (12.6 hours) indicate IPTG addition for inducing protein expression and a first lactose addition. Vertical, dotted lines (20.5 hours) indicate the end of the batch phase and the start of galactose and nitrogen additions. a) Profile of the total carbon source added to the system: galactose (solid line), nitrogen source: ammonium phosphate (dotted line) and lactose (dashed line); b) cell dry weight concentration (CDW); c) Concentrations of LNT II (open triangles) and LNT (solid circles).

FIG. 11: Structure of fucosylated lacto-N-triose II.

FIG. 12: Structure of difucosylated lacto-N-pentose.

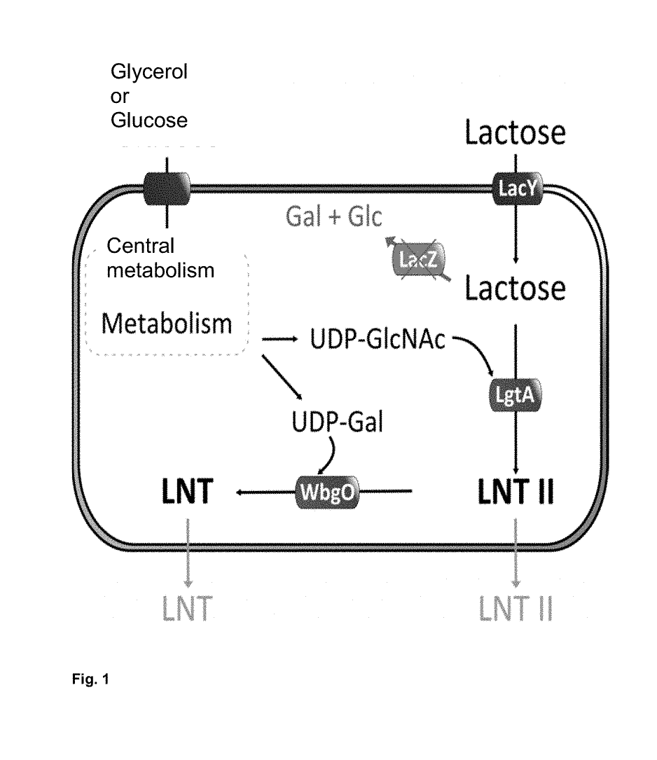

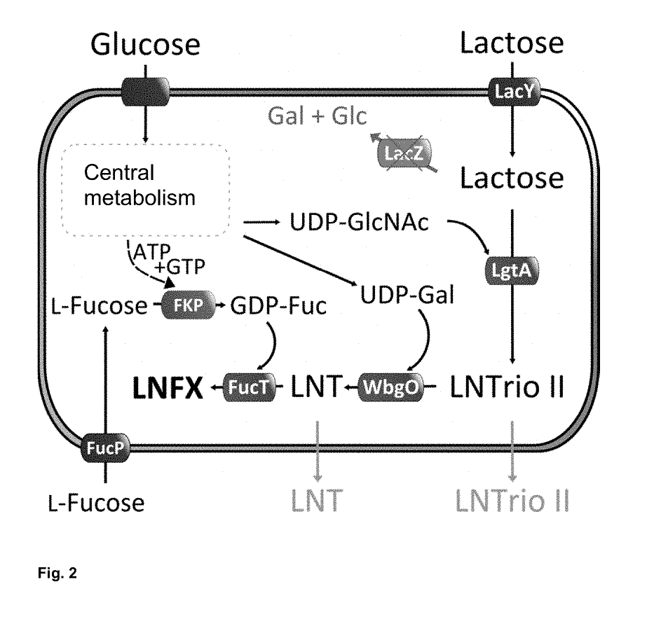

### EXAMPLE 1

### Preparation of a Genetically Modified Microorganism of the Invention

The starting strain for said preparation was the E. coli K-12 strain LJ110. This plasmid-free strain was modified by knocking out sugar breakdown gene loci in the corresponding expression cassettes by means of homologous recombination. The β-galactosidase-encoding lacZ gene was removed and the strain was provided with the IgtA gene coding for Neisseria meningitidis β1,3-N-acetylglucosaminyltransferase to allow synthesis of LNT II. Finally, the strain was furnished with the wbgO gene coding for the WbgO β1,3-galactosyltransferase. Said genes were integrated chromosomally.

This involved firstly cloning the IgtA gene into an expression vector having an IPTG-inducible P_{tac} promoter, which expression vector was then furnished with an FRT-flanked chloramphenicol resistance gene downstream of the IgtA gene. The expression cassette including P_{tac} promoter, a ribosome binding site (Shine-Dalgarno sequence), IgtA, FRT-cat-FRT resistance marker, and an rrnB transcription terminator sequence was amplified by means of PCR. The cassette was then chromosomally integrated into the LacZYA locus.

The strain was furthermore provided with an E. coli K12 lacY gene under the control of a P_{tac} promoter to ensure lactose uptake. For this purpose, lacY was cloned into an expression vector followed by generating an appropriately resistance-labeled expression cassette by downstream cloning of an FRT-kan-FRT resistance cassette. After amplification, said expression cassette was chromosomally integrated into the fucIK locus.

For intracellular conversion of LNT II to LNT, the wbgO gene from the E. coli O55:H7 strain, which codes for a β1,3-galactosyltransferase, was chromosomally integrated into the xylAB locus, as described for IgtA.

### EXAMPLE 2

### Investigating the Formation of LNT and LNT II Using Various Carbon Sources

In spite of catabolite repression, described in the literature (see McGinnis et al. J. Bacteriol. 1969, 100, 902-913), by glucose on galactose, this experiment employed galactose both in the mixture with glucose or glycerol and as the sole utilizable carbon source in minimal medium, in order to analyze product formation. The strain prepared in example 1 was used for the experiments. The culture in each case was 50 ml in size. The main carbon sources, glucose, glycerol and galactose, were each used at a final concentration of 10 g I⁻¹, while lactose was used at a final concentration of 2 g I⁻¹, with the admixed galactose likewise being used at 2 g I⁻¹, both being added at the time of induction at OD₆₀₀=0.4-0.6 (with 0.5 mM IPTG, final conc.).

Formation of LNT was determined fluorometrically by means of HPLC both in the culture supernatants and in the culture pellets, 24 hours after induction, after derivatization with anthranilic acid (see Ruhaak et al. Proteomics 2010, 10, 2330-2336). Thus, for example, an improved LNT yield was observed when switching from glycerol to glucose. As expected, addition of galactose to the culture containing glucose showed neither an effect on growth nor on product formation, due to catabolite repression. When galactose was the only carbon source used, apart from lactose, or when galactose was added to the culture containing glycerol at induction, LNT concentration was markedly increased in said cultures 24 hours after induction. Adding galactose to the culture containing glycerol increased LNT concentration by a factor of 2.7 to 434.3 mg I⁻¹, thus exhibiting a rate of product formation of about twice the value when glucose was used. When the culture medium employed 10 g I⁻¹ galactose without glucose or glycerol, an LNT concentration of 798.1 mg I⁻¹ was achieved. Thus, the previously highest value achieved with glucose was increased by a factor of 3.6 (see FIG. 4).

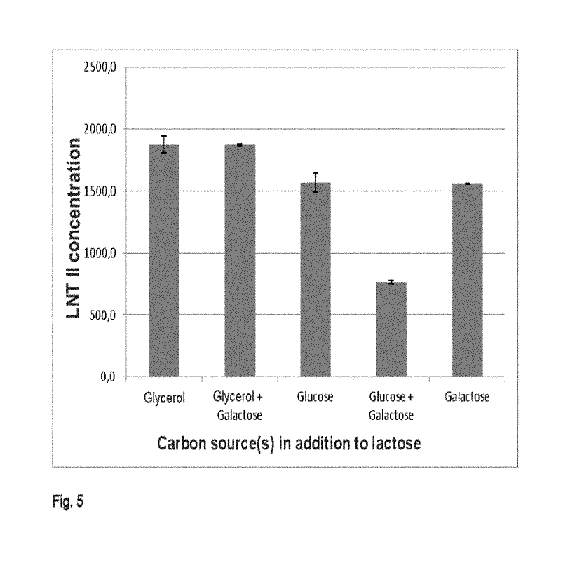

Looking at production of the trisaccharide LNT II reveals that by comparison LNT II synthesis is highest with glycerol as carbon source, while glucose and galactose result in approx. 16.4% less LNT II synthesis. When glucose and galactose are employed together, LNT II concentration 24 h after induction is distinctly lower, at only 769 mg I⁻¹ (see FIG. 5). This may be explained possibly by the lower cell density of the culture. However, an interplay of inducer exclusion by glucose uptake (see Nelson et al., EMBO J. 1983, 2, 715-720) and inhibition of Lac permease by the galactose present (see Olsen et al., J. Biol. Chem. 1989, 264, 15982-15987) is also conceivable, resulting in less lactose being taken up than in systems with only one kind of inhibition of Lac permease. This inhibition is also supported by the fact that the ratio of LNT to LNT II and lactose in the pellet is markedly higher than in the cultures containing other carbon sources.

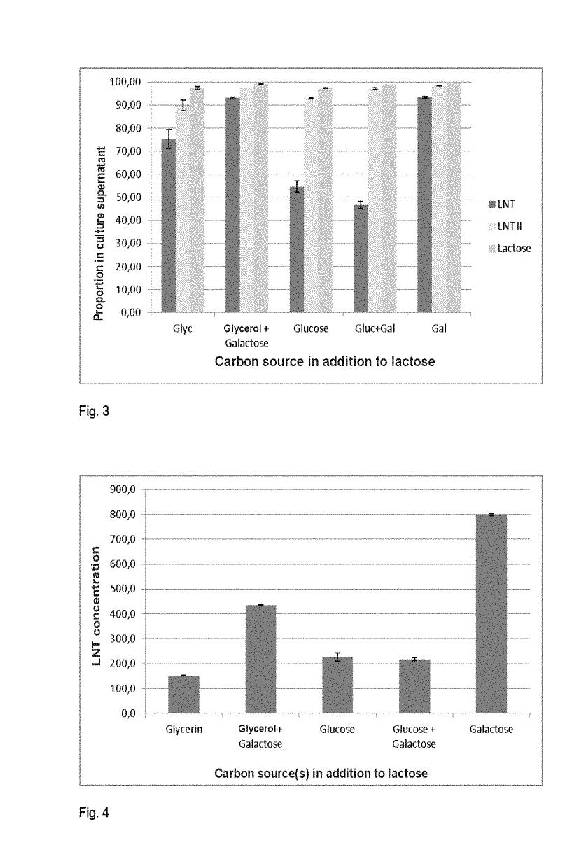

From looking at the proportion of LNT in the culture supernatant (see FIG. 3), it becomes apparent that the strain on galactose not only synthesizes markedly more LNT, but also the proportion of LNT in the culture supernatant, at approx. 93.3%, is significantly higher than the proportion in the culture supernatant of the glucose cultures (approx. 54.6%). Since isolating LNT from the culture supernatant is advantageous compared to isolating from the culture pellet and also more product is formed with galactose as carbon source, synthesis of LNT using galactose is presumably, despite the higher substrate cost, particularly advantageous and therefore preferred.

Formation of the tetrasaccharide LNT requires the transfer of glycosyl from N-acetylglucosaminyl and galactosyl units to the acceptor substrate lactose. The respective donor substrates, UDP-N-acetylglucosamine (UDP-GlcNAc) and UDP-galactose (UDP-Gal), which are required for cytosolic glycosyltransferase reactions, are provided by the E. coli metabolism—as already mentioned at the outset. However, incomplete conversion of lactose to LNT indicates a limited supply of donor substrates, in particular UDP-galactose.

To improve the intracellular availability of UDP-galactose and thus to be able to achieve an increased LNT yield, minimal media containing the various carbon sources were studied further in detail with regard to the conversion of lactose and product formation and also release of the products into the medium. This involved again using the strains prepared according to example 1 which were cultured in minimal media containing one of 1% glucose, 1% glycerol and 1 galactose, or 1% glucose or 1% glycerol supplemented in each case with 0.2% galactose. Expression of the recombinant genes and synthesis of LNT were initiated in each culture by adding IPTG (final concentration of 2 g L⁻¹) to the cells in the early exponential growth phase. The concentrations of lactose, LNT II and LNT were determined in each culture 24 hours post induction.

The strains were cultured in LB medium containing 50 μg mL⁻¹ chloramphenicol (to avoid contamination) at 37° C. A lacto-N-tetrose standard with a purity of more than 95% was obtained from IsoSep (Tullinge, Sweden). Standards of UDP-glucose disodium salt hydrate (≧98%) and UDP-N-acetylglucosamine sodium salt (≧98%) were obtained from Sigma Aldrich (Taufkirchen, Germany), and UDP-galactose disodium salt (≧95%) was obtained from Calbiochem (Merck, Darmstadt, Germany). Lactose monohydrate (Ph. Eur. grade), glucose monohydrate (≧99.5%), glycerol (≧98%) and galactose (≧98%) were obtained from Carl Roth (Karlsruhe, Germany). All other chemicals and reagents were obtained with the highest purity available from either Carl Roth (Karlsruhe, Germany) or Sigma Aldrich (Taufkirchen, Germany).

Synthesis of LNT II and LNT was carried out at 30° C. and 90 rpm in in each case two batches in 500 mL shaker bottles containing 50 mL minimal medium comprising 1% of the main carbon source (glycerol, glucose or galactose) and chloramphenicol (50 μg ml⁻¹, to avoid contamination). The medium had the following composition: 2.68 g L⁻¹ (NH₄)₂SO₄, 1 g L⁻¹ (NH₄)₂—H citrate, 10 g L⁻¹ main carbon source (glycerol, glucose or galactose), 14.6 g L⁻¹ K₂HPO₄, 0.241 g L⁻¹ MgSO₄, 10 mg L⁻¹ MnSO₄.H₂O, 2 g L⁻¹ Na₂SO₄, 4 g L⁻¹ NaH₂PO₄.H₂O, 0.5 g L⁻¹ NH₄Cl, 10 mg L⁻¹ thiamine hydrochloride, and trace solution (3 mL L⁻¹: 0.5 g L⁻¹ CaCl₂.2H₂O, 16.7 g L⁻¹ FeCl₃.6H₂O, 20.1 g L⁻¹ Na₂-EDTA, 0.18 g L⁻¹ ZnSO₄.7H₂O, 0.1 g L⁻¹ MnSO₄.H₂O, 0.16 g L⁻¹ CuSO₄.5H₂O, and 0.18 g L⁻¹ CoCl₂.6H₂O). The cultures were inoculated with a single colony grown on minimal medium agar plates containing 1% of the corresponding carbon source. After reaching an optical density at 600 nm (OD₆₀₀) of 0.4-0.6, the cultures were induced with 0.5 mM IPTG (final concentration), with 2 g L⁻¹ lactose being added at the time of induction. To determine the galactose, lactose, LNT II and LNT levels, 2 mL samples were centrifuged (15300 g, 2 min) 24 hours post induction. After centrifugation the supernatants were stored at −20° C. until derivatization; the pellets were washed with 1 mL of ice-cold saline, centrifuged as before, and likewise stored at −20° C.

The cell dry weights (CDWs) of the cultures containing one main carbon source (glycerol, glucose or galactose) were analyzed by centrifugation (5869 g, 4° C., 20 min) of 10 mL of culture and drying of the cell pellet at 120° C. to constant weight (minimum of the two batches), in each case 24 hours post induction. CDW [g L⁻¹] to OD₆₀₀ [−] correlations were determined in shaker bottles (0.3 for glycerol as main carbon source, 0.37 for glucose as main carbon source, and 0.39 for galactose as main carbon source).

As FIGS. 8a and 8b show, the carbon source provided has a significant influence on the conversion of lactose, with the results indicating that the carbon sources used affect both the shift toward more UDP-activated sugars and lactose uptake. Whereas cultures grown on glycerol, as described above, resulted in the lowest LNT yield (0.152±0.002 g L⁻¹), using a glycerol plus galactose mixture increased the LNT yield in turn by a factor of nearly 3. In contrast, the comparison of cultures grown on glucose or on a glucose/galactose mixture showed about the same yields of LNT, but conversion of lactose to LNT II was significantly lower in the case of the mixture. The highest conversion of lactose, as well as the highest LNT yield (0.810±0.013 g L⁻¹), were observed in cultures which had grown on galactose only.

In addition to influencing conversion of lactose to LNT II and LNT, the carbon sources used also showed an effect on releasing the product LNT (see FIG. 8c). While cultures containing glucose or a glucose/galactose mixture resulted in a release of about 50% of the LNT produced, more than 90% of the LNT formed were found in the culture medium in cultures containing galactose or glycerol/galactose.

The shaker bottle experiments showed that the carbon source can apparently influence formation of LNT. To determine whether the carbon sources used can control intercellular availability of the donor substrates and thus product formation, the concentrations of UDP-N-acetylglucosamine, UDP-glucose and UDP-galactose were quantified. This involved culturing the strain prepared according to example 1 in minimal medium with one of glycerol, glucose and galactose, harvesting the cells in the late exponential growth phase, and analyzing the intercellular metabolites by HPLC.

The strain was cultured at 30° C. and 90 rpm in 1 L shaker bottles charged with 100 mL of minimal medium containing glycerol, glucose or galactose, as described above. At OD₆₀₀ 0.4-0.6, expression was induced with 0.5 mM IPTG and the cultures were incubated further at 30° C. and 90 rpm. Twelve hours post induction, 25 ml samples were centrifuged (2876 rpm, 4° C., 15 min). The pellets were subsequently resuspended in quenching buffer (acetonitrile:methanol:H₂O 4:4:2 with 0.1 M formic acid (Bennett et al. Nat. Chem. Biol., 2009, 5, 593-599)) and mixed vigorously at 4° C. on a Vortex mixer every 3 minutes during incubation on ice for 10 min, and the suspension was then neutralized with 1 M NH₄OH. The samples were then centrifuged again (22410 g, 4° C., 10 min). The supernatants were dried in a Speedvac CON-1000 (Frobel, Lindau, Germany) and dissolved in H₂O in 5% of the extraction volume prior to HPLC analysis. The UDP sugars were analyzed using a Dionex HPLC instrument (Thermo Fisher Scientific, Dreieich, Germany) equipped with Chromeleon software, a Gina autosampler, P580 pumps, a UVD diode array detector and a Luna C18(2) reverse phase column (250 mm×4.5 mm, 5 μm, Phenomenex, Aschaffenburg, Germany). The following gradient, modified from Payne and Ames (Anal. Biochem., 1982, 123, 151-161), was applied at a flow rate of 1 mL min⁻¹: 0 to 30 min linear gradient from 100% solvent NO% solvent B to 80% solvent A/20% solvent B, 30 to 30.5 min linear gradient from 80% solvent A/20% solvent B to 100% solvent A/0% solvent B, 30.5 to 35 min isocratic conditions with 100% solvent A to equilibrate the column for the next sample. Identification and quantification were analyzed at 262 nm by comparing the retention times, spectra and signal areas with the commercial standards at seven different concentrations.

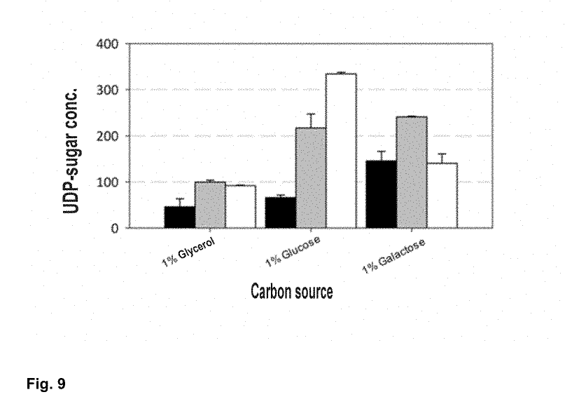

The result revealed that the concentration of UDP-hexoses does indeed significantly depend on the carbon source used. Growth on galactose only resulted in the highest intracellular amount of UDP-galactose (145.63±20.52 nmol L⁻¹ OD⁻¹), approximately 3 times higher than that observed for growth on glucose (65.73±5.63 nmol L⁻¹ OD⁻¹) or glycerol (45.87±17.42 nmol L⁻¹ OD⁻¹). The highest amount of UDP-N-acetylglucosamine was observed during growth on glucose (334.03±3.41 nmol L⁻¹ OD⁻¹) (see FIG. 9).

### EXAMPLE 3

### Demonstration of Scalability of LNT Synthesis on Galactose

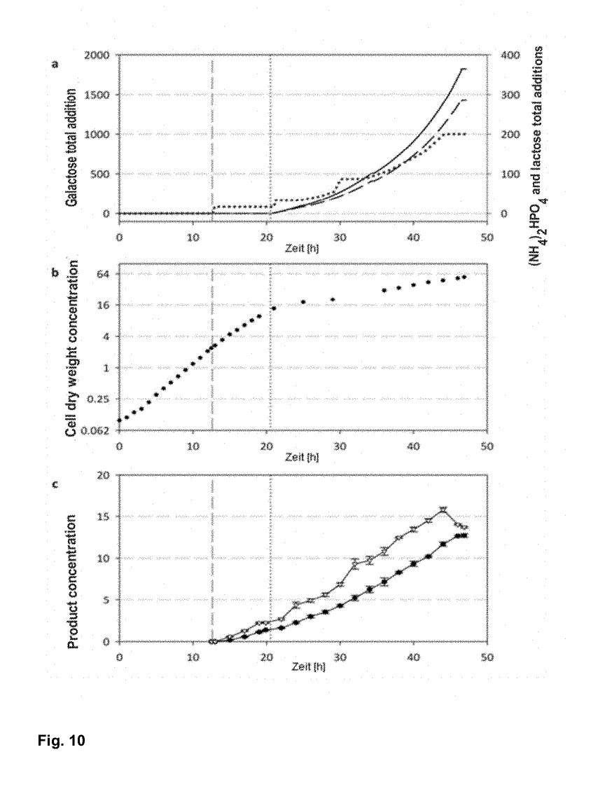

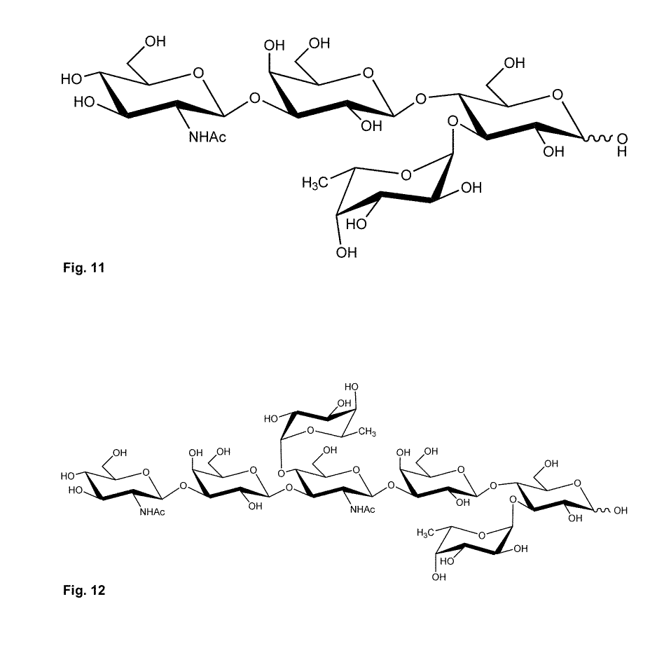

The use of galactose as carbon source for whole cell synthesis of LNT has an advantage in comparison with the E. coli carbon sources normally used, such as glucose or glycerol, due to the higher intracellular UDP-galactose concentration. To demonstrate the scalability of LNT synthesis on galactose, a fed batch cultivation (fed batch process) was carried out in a bioreactor for high cell densities on a 10-liter scale. The process was initiated using an 8.45-liter batch, reaching a biomass concentration of about 13 g L⁻¹ CDW after the galactose initially present had been utilized. During the subsequent feed-in phase, the galactose feed was set so as to maintain a constant growth rate (μ=0.054), resulting in a final biomass of 55.7 g L⁻¹ CDW after 47 hours (see FIGS. 10a,b).

Fed batch cultivation of the strain prepared in example 1 was carried out using mineral salt medium and galactose as main carbon source in a 30 L stirred tank reactor (Bioengineering, Wald, Switzerland) at 30° C., with a starting volume of 8.45 L and a final volume of 13.63 L. The medium was modified from Wilms et al. (Biotechnol. Bioeng., 2001, 73, 95-103) and had the following composition: the eight liters of batch medium consisted of 2.68 g L⁻¹ (NH₄)₂SO₄, 1 g L⁻¹ (NH₄)₂—H citrate, 25 g L⁻¹ galactose, 3.9 g L⁻¹ (NH₄)₂HPO₄, 14.6 g L⁻¹ K₂HPO₄, 0.241 g L⁻¹ MgSO₄, 10 mg L⁻¹ MnSO₄.H₂O, 2 g L⁻¹ Na₂SO₄, 4 g L⁻¹ NaH₂PO₄.H₂O, 0.5 g L⁻¹ NH₄Cl, 10 mg L⁻¹ thiamine hydrochloride, and trace solution (3 mL L⁻¹, composition as described above). During the batch and feed-in phases the pH was regulated by titration with ammonia (25%) to 7.0. Relative dissolved oxygen (pO₂) was maintained above 40% by aeration and agitation, with a reactor pressure of 500 hPa above atmospheric pressure. The batch medium was inoculated with 0.45 L of an overnight preculture to give a cell dry weight concentration of 0.096 g L⁻¹, and cultured at 30° C. and 90 rpm in said mineral salt medium containing 10 g L⁻¹ galactose, as described above for the shaker bottles. Expression of the recombinant genes was induced by adding IPTG (0.5 mM final concentration) 12.6 hours post inoculation, with a cell dry weight concentration of approx. 2.4 g L⁻¹. Lactose (16.9 g) was added at the same time to allow product formation. After the galactose initially provided had been utilized (indicated by a pO₂ increase), the feed-in phase was started with three additions: addition 1 consisted of 514.76 g L⁻¹ galactose, 15.21 g L⁻¹ MgSO₄.7H₂O, 0.65 g L⁻¹ thiamine hydrochloride, and 100.89 ml L⁻¹ trace element solution (composition as described above), while addition 2 consisted of 335.59 g (NH₄)₂HPO₄ and addition 3 consisted of 150 g L⁻¹ lactose for product formation. Additions 1 and 2 were added at a ratio of 81:19, with a galactose-limited growth rate according to formula (1),

$$F ( t ) = [ ( \frac{{μ}_{set}}{{Y}_{\frac{x}{s}}} ) + m ] \times [ \frac{{c}_{x 0} \times {V}_{0}}{{c}_{s 0}} ] \times {e}^{{μ}_{set} \times t} \qquad ( 1 )$$

where F [L h⁻¹] is the rate of addition, t [h] is the feed-in phase time, μₛₑₜ [h⁻¹] is the desired growth rate (fixed at 0.1 in this formula), Yₓₗₛ [g g⁻¹] is the specific yield coefficient of the biomass from the substrate (taken as 0.36 from previous shaker bottle experiments), m [g g⁻¹ h⁻¹] is the specific constant hold coefficient (taken as 0.04), cₓ₀ [g L⁻¹] is the biomass concentration at the start of the feed-in phase (12.0 in this process), V₀ [L] is the culture volume at the start of the feed-in phase (fixed at 8.25), and cₛₒ [g L⁻¹] is the galactose concentration of addition 1 (fixed at 514.76) (Wenzel et al., Appl. Environ. Microbiol., 2011, 77, 6419-6425). Lactose addition was manually adjusted based on utilization, with a total 200.4 g of lactose being added to the system. Cell growth was determined by measuring OD₆₀₀ and calculation of CDW concentration via the correlation factor of 0.47 g I⁻¹ (determined during fermentation) up to a culture density of 40 OD units. CDW concentrations were then determined directly in duplicate by centrifuging 10 mL of culture and subsequently drying the cell pellets to constant weight in glass tubes.

All of the lactose added was utilized and reacted to give LNT II and LNT during cultivation. Both the LNT II and LNT concentrations increased during the process, with final yields of 12.72±0.21 g L⁻¹ (LNT) and 13.70±0.10 g L⁻¹ (LNT II) respectively being reached. The highest LNT II concentration was reached after 44 hours (15.78±0.29 g L⁻¹) and fell subsequently because of lactose utilization and dilution due to the addition of galactose (see FIGS. 10a,c). To ensure complete utilization of lactose at the end of the fermentation process, the lactose feed was stopped 44 hours post inoculation. The yield over time of LNT formation was 0.37 g L⁻¹ h⁻¹ and the final amount of LNT produced in the fed batch process was 173.37±2.86 g, with the large majority of products (88.91±0.06% of LNT II and 64.86±0.12% of LNT) being found in the supernatant of the culture.

### EXAMPLE 4

### Synthesis of Fucosylated Oligosaccharides Having an LNT or LNnT Core Structure

To synthesize fucosylated oligosaccharides that possess an LNT or LNnT core structure, the strain prepared in example 1 was furnished with the appropriate fucosyltransferases for GDP-L-fucose synthesis in a recombinant way. While synthesis of the trisaccharide 2′-fucosyllactose still prefers the de novo synthetic pathway of GDP-L-fucose due to the expensive addition of fucose in the salvage synthetic pathway (see Baumgartner et al., Microb. Cell Fact. 2013, 12, 40), said salvage synthetic pathway is preferred for the synthesis of larger oligo-saccharides. The reason for this is that, with penta- and hexasaccharides being ultimately aimed for, the extra costs no longer matter as much and that the salvage synthetic pathway cannot generate any GDP-L-fucose without the addition of fucose, thereby enabling the start of the fucosylation reactions to be controlled better during cultivation. This is intended to prevent fucosylation from commencing already shortly after induction when using identical promoters for all recombinantly introduced genes, with the result that mainly fucosylated lactose is produced. For this, the bifunctional FKP enzyme having L-fucokinase activity and L-fucose-1-phosphate guanylyltransferase activity from Bacteroides fragilis was used (see Coyne et al., Science (80-.). 2005, 307, 1778-1781; WO2010070104 A1). Since the fkp gene on an expression plasmid has previously been confirmed to be functional, it was integrated into the araBAD arabinose degradation locus of the existing strain.

To synthesize fucosylated LNTs, the strain was transformed with plasmids containing the genes futC (for α1,2-fucosylation, see Albermann et al., Carbohydr. Res. 2001, 334, 97-103) and fucT14 (for α1,4-fucosylation, see Rabbani et al. Glycobiology, 2005, 15, 1076-83; Rabbani et al., Biometals, 2009, 22, 1011-7, not described previously for in vivo applications in E. coli), respectively, or with the corresponding empty plasmid. To synthesize fucosylated LNnTs, the strain used was likewise transformed with plasmids comprising the genes futC and futA (for α1,3-fucosylation, see Ge et al. J. Biol. Chem. 1997, 272, 21357-63), respectively. To check product formation, the strains were cultured in each case in minimal medium containing glucose (10 g I⁻¹) and casamino acids (1 g I⁻¹ final conc., Difco, for more reliable growth) in shaker flasks. This involved inducing protein expressions in each case at OD₆₀₀=0.4-0.6 with IPTG (0.5 mM final conc.) and adding lactose (2 g I⁻¹ final conc.) at the same time. Fucose (2 g I⁻¹ final conc.) was added 26 hours after induction with IPTG and after prior sample taking and addition of 0.5 culture volumes of minimal medium containing glucose (10 g I⁻¹) to ensure continued supply with enough carbon and the preceding synthesis of sufficient amounts of LNT or LNnT. The cultures were then incubated further at 30° C. and 90 rpm, and a second sample taking was carried out 65 hours after the first induction. When compared with the control with empty plasmid after 65 hours, the strains showed in each case products which can be attributed to the successful fucosylation of the core structures.

### EXAMPLE 5

### Synthesis of Fucosylated LNT or LNnT Core Structures Using Strains in which the Fucosyltransferase Genes were Placed Under the Control of a Rhamnose-Inducible Promoter

The same experiments were carried out again using strains in which the fucosyltransferase genes are not located on vectors having an IPTG-inducible tac promoter, but in plasmids in which the fucosyltransferase genes (futC) have been placed under the control of a rhamnose-inducible promoter (see Wiese, A. Molekulargenetische and funktionelle Charakterisierung des Hydantoin-Operons aus Arthrobacter aurescens DSM 3747 [Molecular genetic and functional characterization of the hydantoin operon from Arthrobacter aurescens DSM 3747]. (2000)). This involved inducing expression of the fucosyltransferase genes first with L-rhamnose (2 g I⁻¹) as well as adding L-fucose to provide more protein formation resources for the LgtA and LgtB/WbgO glycosyltransferases. The shaker flask cultures which were otherwise carried out in a manner similar to the previous experiments exhibited stronger signals with fucosyltransferases in the samples at 65 h post induction. For antibiotics-free synthesis of these structures, the fucosyltransferase genes were also chromosomally integrated into the rhaBAD rhamnose operon. The integrated fucosyltransferase genes are in each case under the control of a tac promoter here. All strains in shaker flask experiments were shown by means of HPLC and mass spectrometry to have the ability to synthesize fucosylated LNT or LNnT.

### EXAMPLE 6

### Synthesis and Isolation of Larger Fucosylated Oligosaccharides

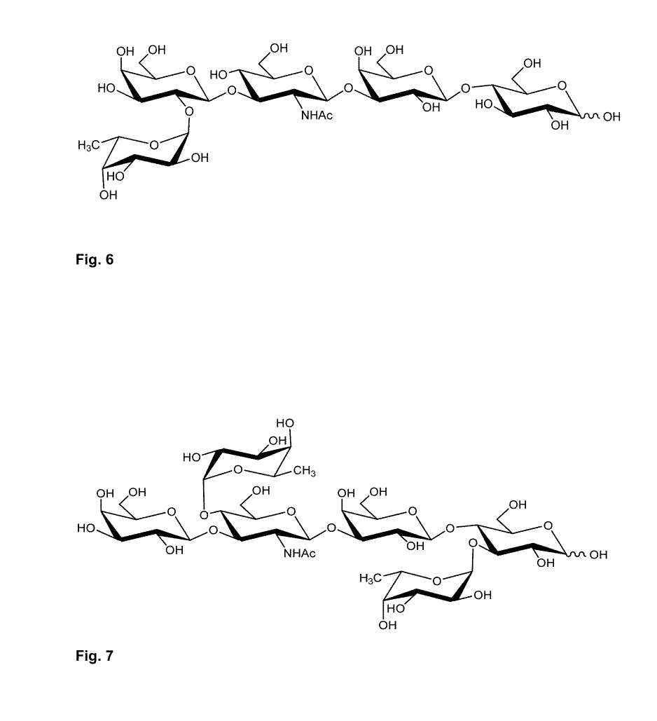

Since the experiments stated above demonstrated also for LNnT that the fucosylations work, and since α1,3- and α1,2-fucosylated compounds based on LNnT have already been produced (see Drouillard et al., Angew Chem Int Ed Engl 2006, 45, 1778-1780; Dumon et al., Glycoconj. J. 2001, 18, 465-474), the fucosylated compounds produced were to be isolated and further characterized. To this end, the above cultivations containing the L-rhamnose-inducible plasmids were repeated on a 750 ml scale in 3-liter shaker flasks with baffles under otherwise identical conditions as before. The products were then recovered from the cell pellets by resuspension in H₂O, incubation at 100° C. for 20 minutes. and centrifugation and subsequently isolated by means of preparative activated carbon/Celite545 chromatography and gel filtration chromatography. This involved employing in addition to Bio-Gel P2 from Bio-Rad also Bio-Gel P4 (extra fine) with a narrower particle size distribution and a larger cut-off, since the latter has already been used successfully for fractionating larger neutral oligosaccharides (see Priem et al., Glycobiology 2002, 12, 235-240). These isolating steps were able to isolate from the cultures 59.4 mg of LNFI in total (for structure see FIG. 6). Parts thereof were studied by means of mass spectrometry and NMR, in order to fully elucidate the structure. The mass spectrum of the product here shows, apart from a few contaminations, especially the signals of the proton adduct, sodium adduct and disodium adduct of LNFI.

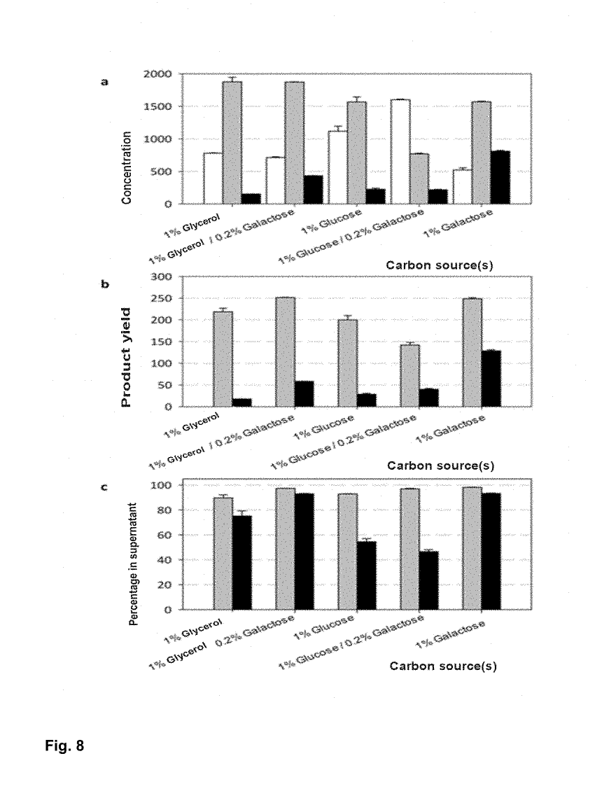

Oligosaccharide isolation from a strain furnished with FucT14 produced 133.7 mg of LNDII in total (for structure, see FIG. 7). Moreover, 71.5 mg of fucosylated lacto-N-triose II were isolated. The mass spectra here likewise showed the masses of the expected adducts and hardly any contaminations. The substances are fucosylated or difucosylated compounds with LNT as core structure, which have not been described previously as compounds synthesized in vivo in E. coli (nor are other synthesis pathways with similar amounts of product known). Another compound appearing in the isolation process is a lacto-N-pentose with two fucosyl residues which is probably the result of elongation of LNT with another N-acetylglucosaminyl group.

1-17. (canceled)

18. A genetically modified microorganism for in vivo synthesis of lacto-N-tetrose or lacto-N-neotetrose, said microorganism comprising:
(i) a first transgene coding for β1,3-N-acetylglucosaminyltransferase; and
(ii) a second transgene coding for β1,3-galactosyltransferase or β1,4-galactosyltransferase.

19. The genetically modified microorganism of claim 18, wherein said microorganism is further genetically modified to suppress expression of LacZ and LacA.

20. The genetically modified microorganism of claim 18, wherein the first transgene is integrated into the LacZYA locus and the microorganism comprises a further transgene coding for LacY.

21. The genetically modified microorganism of claim 20, wherein the transgene coding for LacY is integrated into the FucIK locus.

22. The genetically modified microorganism of claim 18, wherein said microorganism comprises a further transgene coding for UDP-sugar pyrophosphorylase.

23. The genetically modified microorganism of claim 18, wherein said microorganism is further genetically modified to suppress expression of UDP-glucose 4-epimerase.

24. The genetically modified microorganism of claim 18, wherein one or both, or one, multiple or all, transgenes is/are chromosomally integrated.

25. The genetically modified microorganism of claim 18, wherein said microorganism comprises a further transgene coding for a bifunctional enzyme having L-fucokinase activity and L-fucose-1-phosphate guanylyltransferase activity, and at least one transgene coding for an enzyme capable of alpha1,2-fucosylation, alpha1,3-fucosylation or alpha1,4-fucosylation.

26. The genetically modified microorganism of claim 25, wherein the transgene coding for a bifunctional enzyme having L-fucokinase activity and L-fucose-1-phosphate guanylyltransferase activity is chromosomally integrated, and the at least one transgene coding for an enzyme capable of alpha1,2-fucosylation, alpha1,3-fucosylation or alpha1,4-fucosylation is expressed on a plasmid vector.

27. The genetically modified microorganism of claim 25, wherein both the transgene coding for a bifunctional enzyme having L-fucokinase activity and L-fucose-1-phosphate guanylyltransferase activity and the at least one transgene coding for an enzyme capable of alpha1,2-fucosylation, alpha1,3-fucosylation or alpha1,4-fucosylation are chromosomally integrated.

28. The genetically modified microorganism of claim 18, wherein the microorganism is selected from the group consisting of bacteria, fungi, and plants, or wherein the microorganism is of the genera Corynebacterium, Bevibacterium, Bacillus, Saccharomyces, or Escherichia.

29. A method for in vivo synthesis of lacto-N-tetrose or lacto-N-neotetrose, or a fucosylated derivative of lacto-N-tetrose or lacto-N-neotetrose, comprising utilizing the genetically modified microorganism of claim 18.

30. A method of preparing lacto-N-tetrose or lacto-N-neotetrose, or a fucosylated derivative of lacto-N-tetrose or lacto-N-neotetrose, comprising:
(a) providing the genetically modified microorganism of claim 18;
(b) culturing said genetically modified microorganism under conditions that permit synthesis of lacto-N-tetrose or lacto-N-neotetrose;
(c) optionally adding fucose; and
(d) optionally isolating lacto-N-tetrose or lacto-N-neotetrose, or fucosylated derivative of lacto-N-tetrose or lacto-N-neotetrose.

31. The method of claim 30, wherein step (b) comprises:
(i) using galactose as carbon source for said microorganism; or
(ii) using glycerol and galactose as carbon source for said microorganism.

32. The method of claim 30, wherein step (b) comprises adding one or more carbon sources continuously or in batches.

33. The method of claim 32, wherein said one or more carbon sources are selected from the group consisting of lactose, glucose, glycerol, galactose, and any mixtures thereof.

34. The method of claim 30, wherein said method is carried out by way of a fed batch process with a batch volume in the range from 2 to 30 L.

35. The method of claim 30, wherein said method is carried out by way of a fed batch process with a batch volume in the range from 3 to 20 L.

36. The method of claim 30, wherein said method is carried out by way of a fed batch process with a batch volume in the range from 5 to 15 L.

37. The method of claim 30, wherein the microorganism is selected from the group consisting of bacteria, fungi, and plants, or wherein the microorganism is of the genera Corynebacterium, Bevibacterium, Bacillus, Saccharomyces, or Escherichia.
